(19) United States
(12) Patent Application Publication
(10) Pub. No.: US 2026/0232592 A1
(43) Pub. Date: Aug. 13, 2026

(54) GLUTATHIONE-COATED GOLD NANOPARTICLES FUNCTIONALIZED WITH LITHIUM (LiG-AuNPs) AND USES THEREOF FOR THE MODULATION OF GLYCOGEN SYNTHASE KINASE - 3 ACTIVITY

(71) Applicants: UNIVERSITÀ CATTOLICA DEL SACRO CUORE, Milano (IT); FONDAZIONE POLICLINICO UNIVERSITARIO AGOSTINO GEMELLI IRCCS, Rome (IT); UNIVERSITÀ DEGLI STUDI DI SALERNO, Fisciano (IT)

(72) Inventors: Roberto Piacentini, Milano (IT); Claudio Grassi, Milano (IT); Antonio Buonerba, Fisciano (IT); Alfonso Grassi, Fisciano (IT)

(73) Assignees: UNIVERSITÀ CATTOLICA DEL SACRO CUORE, Milano (IT); FONDAZIONE POLICLINICO UNIVERSITARIO AGOSTINO GEME, Rome (IT); UNIVERSITÀ DEGLI STUDI DI SALERNO, Fisciano, SA (IT)

(21) Appl. No.: 19/155,034

(22) PCT Filed: Feb. 1, 2024
(86) PCT No.: PCT/IB2024/050915

§ 371 Date: Aug. 8, 2025

(30) Foreign Application Priority Data
Feb. 10, 2023 (IT) 102023000002310

(51) Int. Cl. A61K 9/16; A61K 33/00; A61K 47/02; A61K 47/42

(52) U.S. Cl. CPC A61K 9/1676; A61K 9/1682; A61K 33/00; A61K 47/02; A61K 47/42

(57) ABSTRACT

The present invention relates to a method for the production of gold nanoparticles (AuNPs) coated with glutathione and Li⁺ ions, hereinafter designated as LiG-AuNPs, to a method for the preparation of aggregates of said nanoparticles and to the use of said nanoparticles, aggregates or compositions thereof which comprise them for therapeutic use. LiG-AuNPs then are an effective instrument in inhibiting GSK-3 and its downstream molecular targets, while keeping the lithium extracellular concentration levels below the systemic toxicity threshold (1.5 mEq/L), and exerting an antioxidant action by means of the glutathione present on their surface.

### FIELD OF THE INVENTION

The present invention relates to a method for the production of gold nanoparticles (AuNPs) coated with glutathione and lithium cations (1) (Li⁺), hereinafter designated as LiG-AuNPs, to a method for the preparation of aggregates of said nanoparticles and to the use of said nanoparticles, their aggregates or compositions which comprise them for therapeutic use.

### STATE OF ART

Lithium is an alkali metal, identified for the first time in 1817 and, already a few years after its discovery, found application as a psychotropic drug for the treatment of the mania in the inorganic form of chloride, carbonate, acetate, citrate or sulphate. Although lithium could act on various molecular targets, its main recognized cellular targets are: 1) magnesium-dependent inositol polyphosphate 1 phosphatase and phosphomonoesterase; ii) the Na⁺/K⁺ ATPase (NKA) pump; iii) the Glycogen Synthase Kinase 3 (GSK-3) enzyme. All these three classes of molecules are also dependent on the magnesium ion(II) (Mg²⁺). It is interesting to note that the Li⁺ ionic radius is similar to that of Mg²⁺ which determines the known diagonal relation between the elements of the periodic table; the Li⁺ cations then can compete with the magnesium(II) binding site. The inositol polyphosphate 1 phosphatase is a molecule involved in the production of inositol triphosphate (IP3), an important intracellular messenger involved in releasing Ca²⁺ from the intracellular deposits; NKA is the main cellular motor for generating ionic gradients (of Na⁺) required for the primary and secondary active transportations through the plasmatic membrane; at last, GSK-3 is an important Ser/Thr kinase involved in several intracellular signalling pathways which adjust various cellular processes including, but not limited to, cellular proliferation and differentiation; apoptosis; immune response. GSK-3 is also actively involved in the onset of neurodegenerative diseases such as Alzheimer disease (AD), and other tauopathies, since it is the main kinase responsible for the hyperphosphorylation of the tau protein, as well as involved in the proteolytic cutting of the amyloid precursor protein (APP) for the formation of β-amyloid peptide, characteristic sign of AD. Besides, other studies demonstrated the role of GSK-3 in the phosphorylation of key proteins for the entry and replication of different viruses in cells, thereamong Herpes Simplex Virus (HSV)-1 (see Murru et al., 2020 and mentioned bibliography) and various coronavirus including SARS-CoV-1 and SARS-CoV-2 (responsible for CoViD-19—Liu et al., 2021).

GSK-3 would seem to be the most important target among the various lithium molecular targets. GSK-3 exists in 2 isoforms, designated α and β, and it is a constitutively active kinase. Phosphorylation of the amino acid serine in position 21 and 9 (pGSK-3^{Ser9/21}) respectively for isoform α and β, represents the main inhibitory and modulatory mechanism of GSK-3 activity. Under physiological conditions, such phosphorylation is borne by protein-kinase B, also called Akt, involved in the intracellular signalling pathway, PI3K-mTOR-Akt. However, GSK-3 can be further activated by tyrosine phosphorylation in position 279/216, respectively for the isoforms a/R, dependent on the tyrosine-kinase Fyn. Several studies (Snitow et al., 2021 and references included herein) show that lithium modulates GSK-3 activity at intracellular level by inducing phosphorylation of Ser9/21. Once entered the cells, thanks to the action of some transporters, such as Na⁺—Li⁺ counter-transporter, or not selective ionic channels, such as the channels for Na⁺, Li⁺ exerts its inhibitory action on GSK-3 by competing with magnesium (II) on its binding site (direct action) or activating Akt (indirect action).

The therapeutic application of lithium are several: i) treatment of pathologies which provide the activation of GSK-3 (and in particular of isoform β) as key molecular mechanism, thereamong the disorders of mood and personality (bipolar disorder); ii) treatment of neurodegenerative diseases (ex: Alzheimer disease and various tauopathies, Parkinson and Huntington diseases, all dependent on GSK-3-mediated phosphorylation of key proteins of the various diseases); iii) treatment of neoplastic diseases and viral infections, including those caused by viruses of Herpes Simplex and breathing viruses such as coronaviruses.

Although the lithium-based drugs, mainly in the formulation of lithium carbonate in tablets, have been used for a long time for the treatment of the disorders of mood and/or cluster headaches, the problem of toxicity of lithium at the effective concentrations for the neuropsychiatric and neurodegenerative disorders remain unsolved. This forces constant monitoring of the lithium level at plasma level which has to be kept, in patients treated with these drugs, in the concentration range of 0.8-1.2 mEq/L. This aspect is particularly limiting if one considers the impossibility of using “off label” in the treatment of all pathologies invalidating for the organism (ex. neurodegenerative diseases and viral infections) which require lithium therapeutic concentrations well above the toxicity limit.

It is important to note that many of these pathologies are characterized even by an intense oxidative stress, which often requires a joint treatment with antioxidants.

The development of new lithium-based drugs, highly effective at lower concentrations than the toxicity levels of this metal, is then a strongly wished objective in the treatment of many human and animal pathologies.

As already highlighted previously, the lithium pharmacological use is strongly limited by its high toxicity. The drugs which determine Li⁺ concentrations higher than 1.5 mEq/L in plasma, in fact, induce important functional problems, especially at level of thyroid and kidney system; at lower concentrations unwished effects were observed in people with predisposing dysfunctions or in fragile subjects due to other pathologies. The lithium-based drugs (for example lithium carbonate in 300-mg tablets) mainly used for the mood disorders are generally taken by oral route and the Li⁺ ion is distributed in the organism by systemic route reaching all organs and tissues, including kidneys and thyroid which are affected particularly by the toxic action of this metal cation. Therefore, the posology of such drugs has to be constantly monitored with the purpose of maintaining a lithium plasmatic concentration ranging between 0.4 and 1.2 mEq/L to guarantee a discrete advantage/damage ratio.

Then, the importance results clear of developing administration systems (drug delivery) which guarantee a distribution of the lithium ion in a site-specific (and not systemic) way and which at the same time guarantee the pharmacological effect by reducing the effective concentration below the toxicity threshold.

### SUMMARY OF THE INVENTION

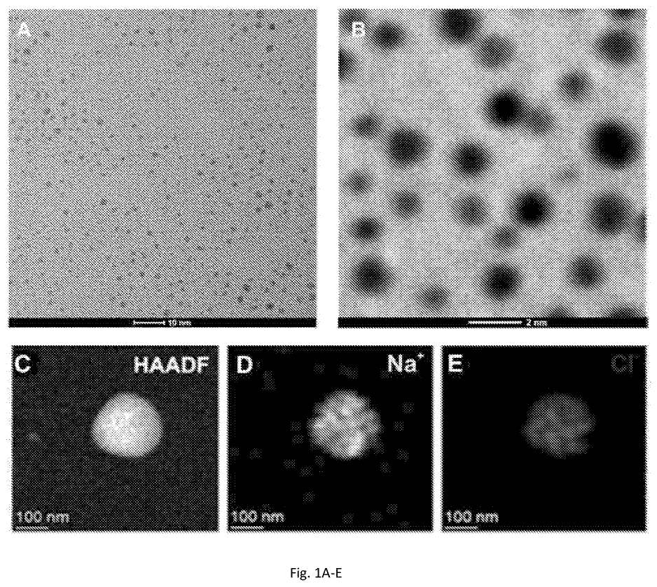

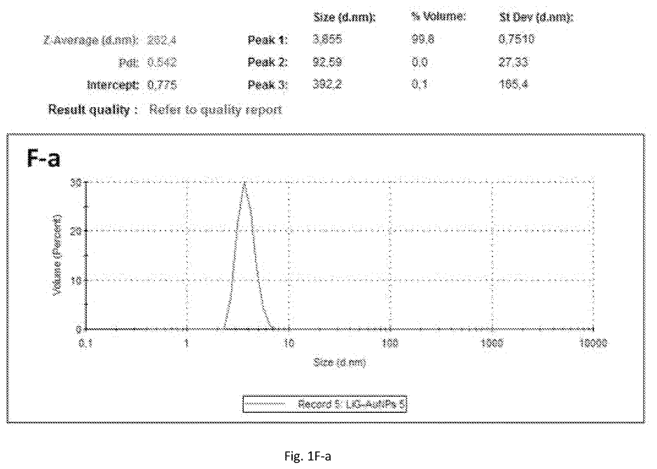

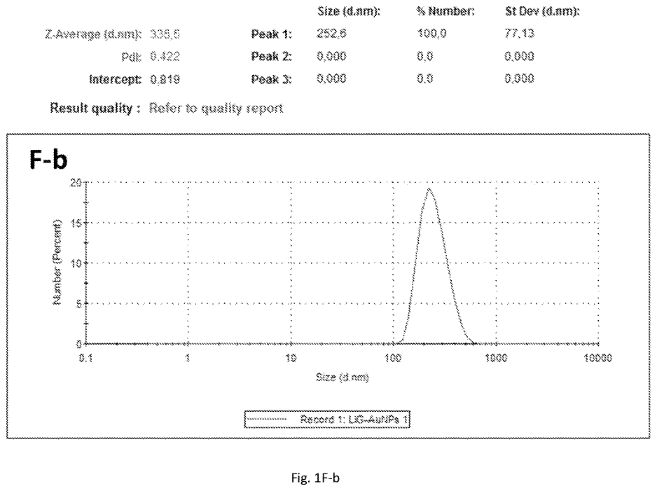

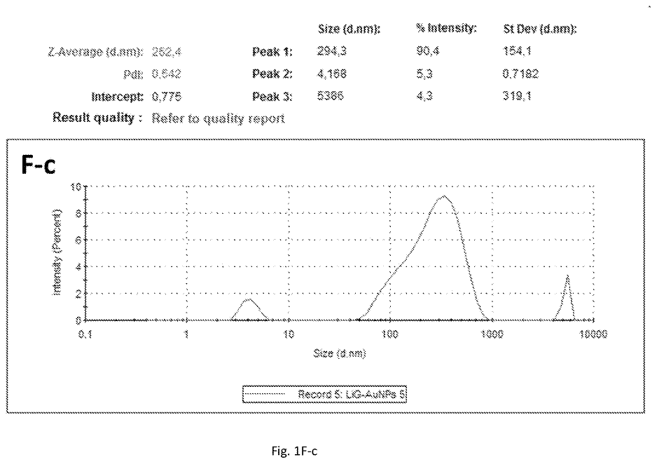

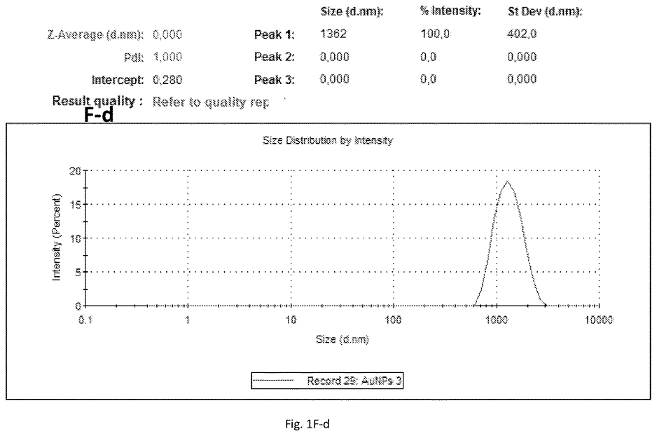

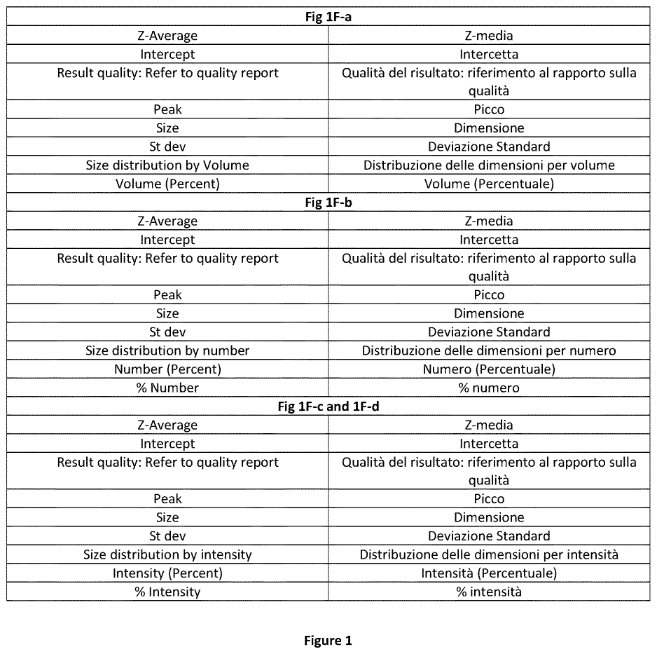

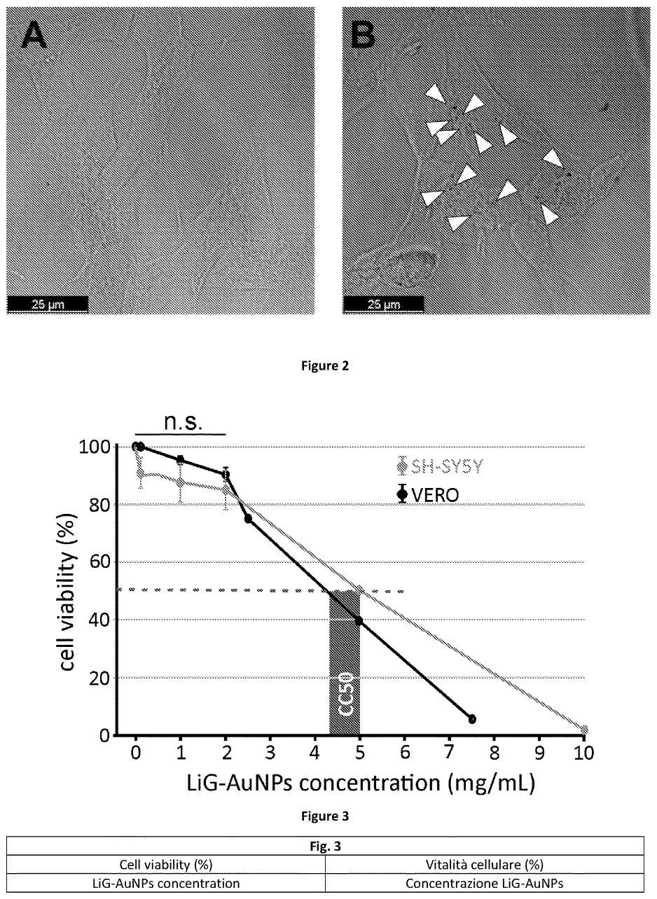

The authors of the present invention have produced gold nanoparticles coated with glutathione and Li⁺ ions which, dispersed in a suitable solvent, form (in the time order of few seconds) aggregates having a diameter of about 100-300 nm, herein also designated as aggregates of LiG-AuNPs (Lithium Glutathione-Gold Nanoparticles) sufficiently stable over time and in a high temperature range, in particular from −20 to 120° C. Only after about 30 days the colloidal aggregates reach sizes of about 1000 nm in diameter (see FIG. 1), which are however equally functional to the purposes described and claimed hereinafter. The authors of the present invention have surprisingly found that the above-mentioned aggregates are rapidly in vitro internalized in human-derived (ex: neuroblastoma or hepatocarcinoma cells) or animal-derived (ex: astrocytes or murine neurons) cells, in a time range comprised between 1 hour and 24 hours (see Buonerba et al., Scientific Reports 2020 and FIG. 2) and which during such internalization process, said aggregates can break down in single nanoparticles with release of Li⁺ ions in the cytosol and/or in the intracellular organelles reached thereby.

A treatment with the above-mentioned aggregates then allows a controlled release of Li⁺ ions, with reduced periods of time, and lower concentrations effective for the pharmacological action than the commonly used lithium salts.

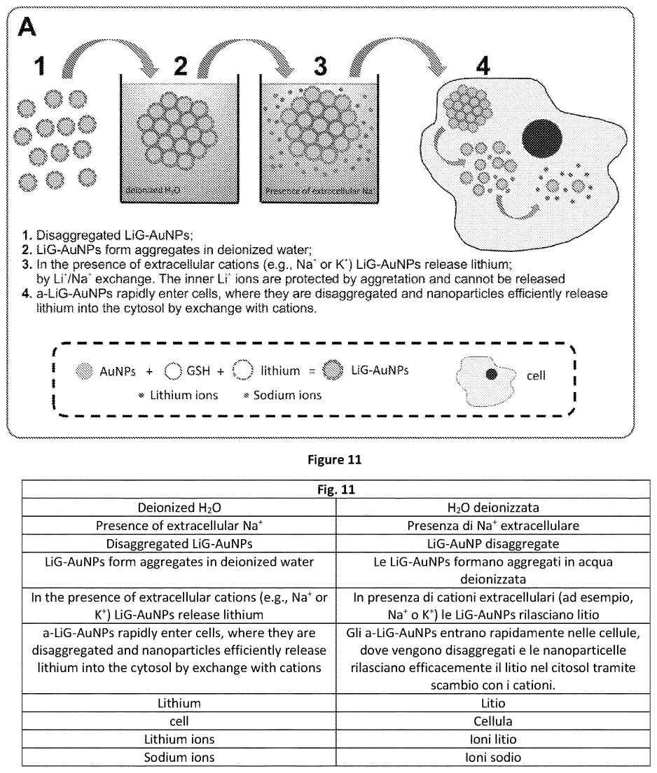

Without wanting to be linked to theory, the authors think that the mechanism for releasing the Li⁺ cation from aggregates and nanoparticles of LiG-AuNPs provides the exchange of this cation with other cations, by way of example, sodium and/or potassium, present in high concentration extra- or intracellularly. In order to support this mechanistic hypothesis the LiG-AuNPs, broken down after sonication, were dispersed in Dulbecco's Modified Eagle Medium (DMEM, standard medium for cellular cultures) where a release in the medium of about 83.5% of the Li⁺ cations in 1 hour was observed, until reaching the value of 91.4% in 24 hours. The residual Li⁺ cations, still present in LiG-AuNPs, probably inside the aggregates, can be internalized in the cells together with the aggregates which, by breaking down inside the cells, determine a more effective intracellular accumulation of lithium than the methods which are based exclusively upon the internalization through non-selective transporters or ion channels (see operation scheme, FIG. 11).

Firstly, the authors of the present invention evaluated the possible in vitro cytotoxicity of LiG-AuNPs. To this purpose, VERO cells (kidney epithelial cells isolated from African green Chlorocebus monkey) and SH-SY5Y cells (human neuroblastoma) were exposed for 24 hours to increasing concentrations of aggregates of LiG-AuNPs dispersed in the culture medium of such cells (from 0.1 mg/mL to 10 mg/mL). For both cellular models, LiG-AuNPs do not determine significative cellular death (vitality 85%) at concentrations≤2 mg/mL, beyond which the cell viability reduces. CC50, concentration at which half of cells die, is estimated at approximately 4.5-5.0 mg/mL in vitro (FIG. 3).

The authors then decided to evaluate the effectiveness of LiG-AuNPs in vehiculating Li⁺ ions inside the cells. To this purpose, SH-SY5Y cells were treated with a non cytotoxic concentration of aggregates of LiG-AuNPs (1 mg/mL, corresponding to 3 mEq/L of Li⁺) for 24 hours and the intracellular concentration of Li⁺ ions was determined through ICP-OES. By comparison other SH-SY5Y cells were treated with LiCl in the culture medium at equal concentration of extracellular Li⁺ (3 mEq/L). In the cells treated with aggregates of LiG-AuNPs, a concentration of intracellular Li⁺ was observed ^{~}26 times more than the one measured in an analogous treatment with lithium chloride, and in particular: 0.154-0.3375 pg_{Li}/cell for the cells treated with LiG-AuNPs and 0.007-0.0375 pg_{Li}/cell in the cells treated with LiCl. It is underlined that the extracellular lithium enters the cells generally by using non-specifically the channels for Na⁺.

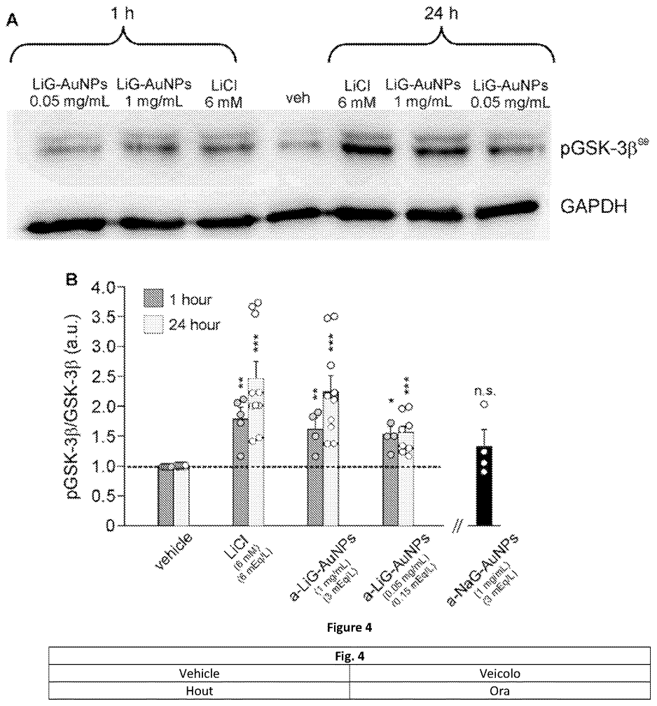

The authors of the present invention further found that the aggregates and the nanoparticles of LiG-AuNPs are particularly effective in inducing significant increases in the inhibitory phosphorylation of GSK-3β (pGSK-3β^{Ser9}), under in vitro experimental conditions. In the specific case, cells of SH-SY5Y human neuroblastoma treated for 1 or 24 hours with aggregates of LiG-AuNPs at concentration of 1 mg/mL (3 mEq/L of Li⁺), show a pGSK-3β^{Ser9}/GSK-3β ratio comparable to the one obtained after a treatment with 6 mM of lithium chloride (corresponding to 6 mEq/L of Li⁺, minimum concentration usually used for in vitro studies of GSK-3 inhibition) (FIG. 4). In order to confirm the effectiveness of LiG-AuNPs in determining a significant increase in pGSK-3β^{Ser9} the SH-SY5Y cells were treated for 24 hours with aggregates of LiG-AuNPs at a usually non effective extracellular concentration of Li⁺ (0.05 mg/mL, corresponding to 0.15 mEq/L Li⁺). Under such condition the pGSK-3β^{Ser9}/GSK-3β ratio resulted to be still significantly higher than the one found in the cells treated with vehicle (+54%±12%; p<1×10⁻³ vs. vehicle) and than those treated for the same time with LiCl 0.15 nM (FIG. 4). At last, in order to confirm the specificity of effect on lithium, some experiments were repeated in SH-SY5Y cells treated for 24 h with analogous nanoparticles functionalized with sodium instead of lithium, that is NaG-AuNPs, at the concentration of 1 mg/mL. Such treatment has no significant effect on the pGSK-3β^{Ser9}/GSK-3β ratio (FIG. 4).

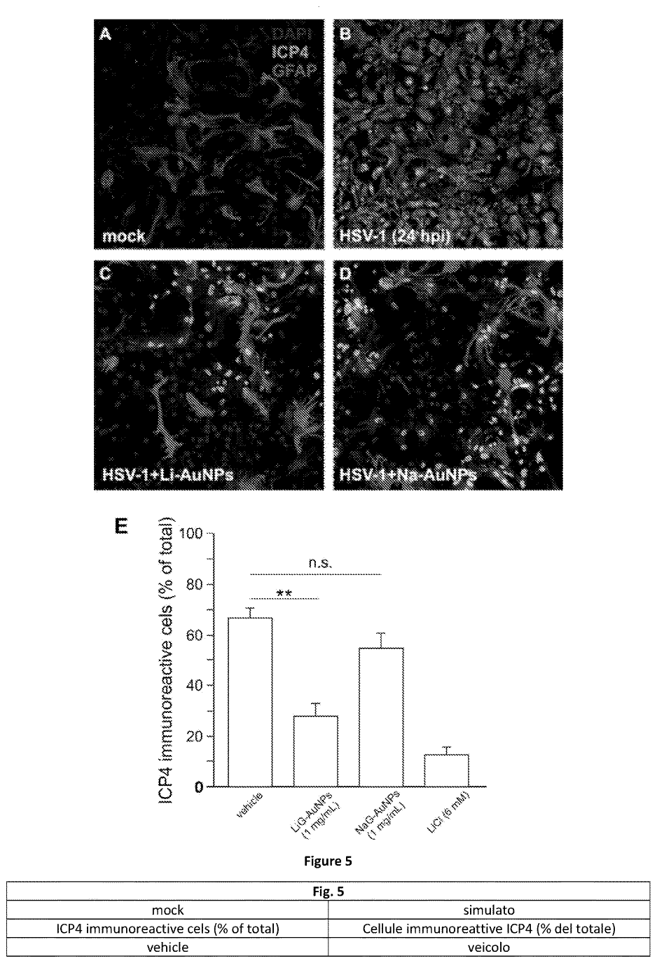

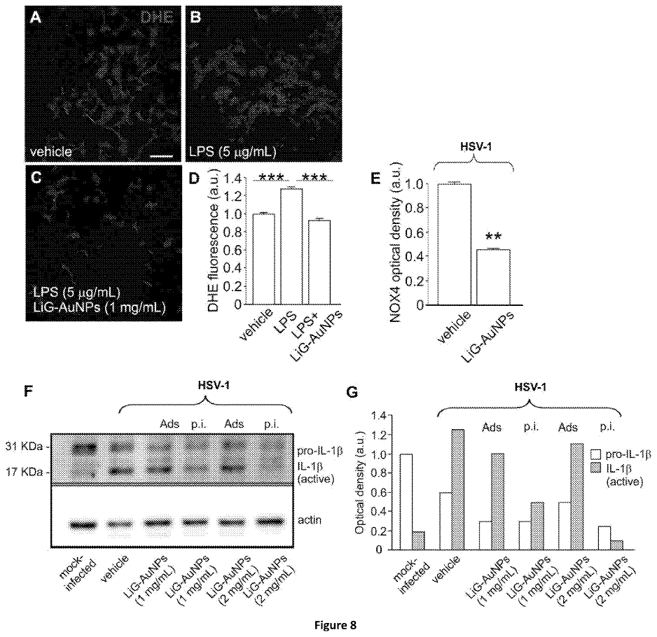

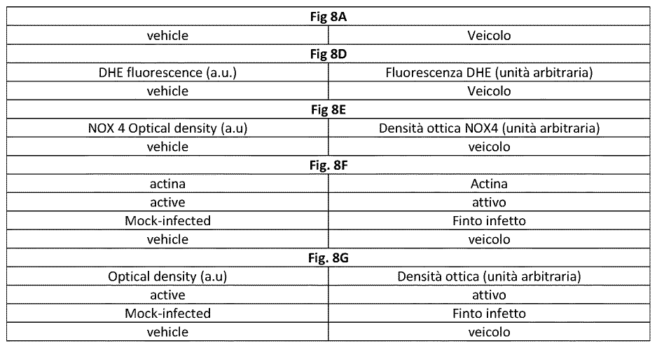

The aggregates and the nanoparticles of LiG-AuNPs, in addition to the above-described modulation of pGSK-3β^{Ser9}, result to be effective against targets downstream of the above-mentioned kinase, thereamong phosphorylation of tau protein and replication of the virus Herpes Simplex type 1 (HSV-1), two events associated therebetween in the nervous cells (De Chiara et al., Plos Pathogens, 2019) by demonstrating the effectiveness in inhibiting the above-mentioned kinase. The replication of HSV-1 is notoriously sensitive to the action of lithium, and depending upon the oxidative stress induced by the viral invasion. In particular, the aggregates and the nanoparticles of LiG-AuNPs demonstrated to be effective in contrasting both the HSV-1 infection and the tau phosphorylation induced by the latter in vitro also at the concentration of 0.05 mg/mL (0.15 mEq extracellular Li⁺), that is at a concentration value widely lower than that usually used for this type of treatments (>10 mM) and above all lower than the toxicity limit of Li⁺ (see examples and FIGS. 5-7). It is important to underline that, advantageously, parallelly to the lithium action, the presence of glutathione on the external crown of nanoparticles counteracts the oxidative stress induced by the viral invasion (see examples and FIG. 8).

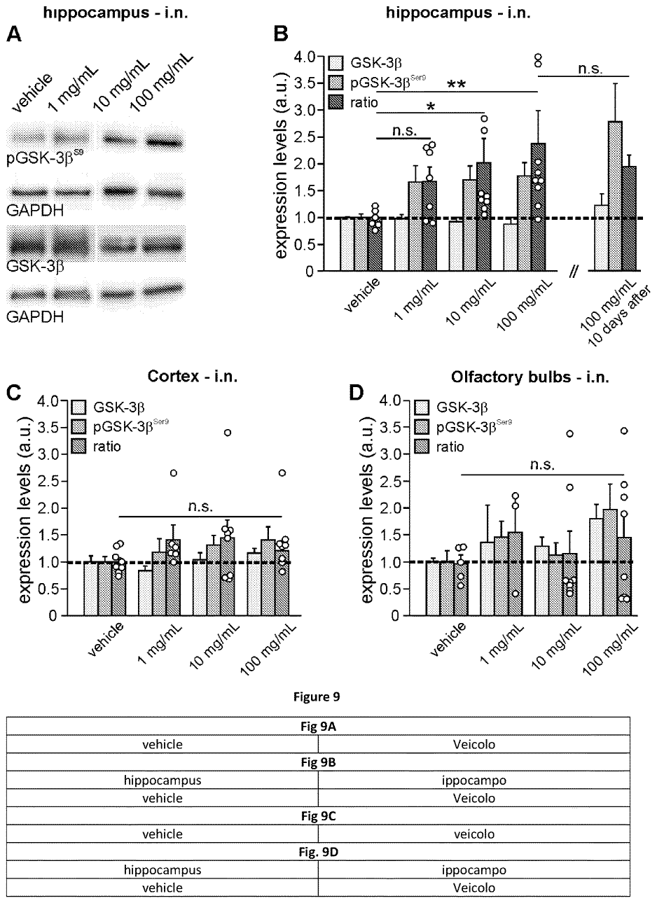

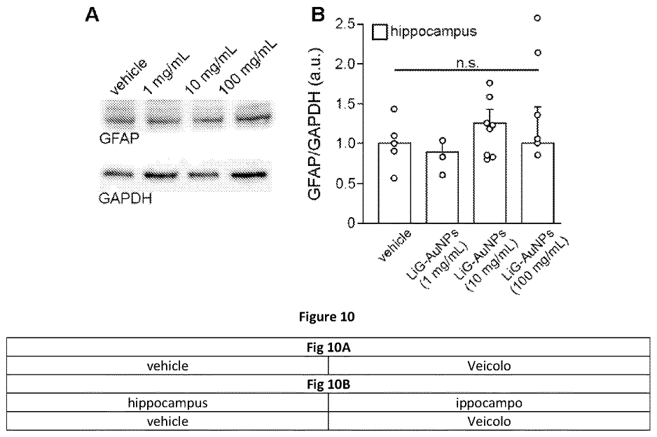

At last, it was demonstrated that, when administered to murine models, LiG-AuNPs easily reach encephalon (as per gold/lithium presence measurements), especially if administered by intranasal route, and once in loco they are capable of modulating the GSK-3 activity, especially at hippocampal level, without determining adverse reactions (for example, gliosis; see section examples and FIGS. 9 and 10).

As shown in details hereinafter, the procedure of synthesis, purification and recovery of LiG-AuNPs is simple, cheap and it does not require the use of sophisticated and expensive equipment. LiG-AuNPs were isolated in solid and sterile form, ready to be redispersed in aqueous and organic means to form aggregates useful to the internalization in target cells. The by-products and the production waste have no problems of toxicity or environment compatibility. At last, the synthesis method is potentially easily scalable to quantities of interest for the pharmaceutical industry of the field.

LiG-AuNPs and their aggregates, the present invention relate to, find application in all biological phenomena modulated by GSK-3 and/or determined by alterations of the intracellular redox status such as, by way of example the neuropsychiatric diseases (ex. mood disorders), neurodegenerative disease (ex. tauopathies), viral infections (ex., HSV-1 o Sars-CoVs) or neoplastic pathologies.

Therefore, the present relates to:

A method for the preparation of glutathione and Li⁺ coated gold nanoparticles, comprising the following steps:
i) preparing a mixture of
   a gold precursor in a concentration from 0.0001M a 10M, preferably from 0.001M to 0.1M, more preferably from 0.010M to 0.020M, still more preferably 0.017M;
   at least a polar solvent;
   glutathione in a molar ratio with respect to said gold precursor from 0.1:1 to 100:0.1, preferably 1:1 to 1:0.1;
   a basic lithium compound in a molar ratio with respect to said gold precursor from 0.01:10 a 10:0.01, preferably 1:1 to 1:0.1, more preferably from 1:0.05 to 1:0.15, still more preferably 1:0.09;
at a temperature from −20° C. to +120° C., preferably at room temperature, in an environment equipped with a stirring and shaking system, to obtain a clear colourless solution;
ii) diluting in a range from 1:0 a 1:1000, preferably from 1:5 to 1:50, more preferably from 1:5 to 1:30, still more preferably 1:20, the solution obtained in step i) with at least a polar solvent;
iii) adding to the diluted solution of step ii) a reducing agent of said gold precursor in a molar ratio with respect to said gold precursor from 1:1 to 100:0.1, preferably 1:1 to 1:0.1, more preferably from 1:0.1 to 1:0.3, still more preferably 1:0.22, to obtain a colloidal suspension of gold nanoparticles, at a temperature from −20° C. to +120° C., preferably at room temperature, in an environment equipped with a stirring and shaking system;
iv) adding a lithium sale in a molar ratio with respect to said gold precursor from 0.1:1 to 100:0.001, preferably from 1:0.001 to 1:1, more preferably from 1:0.001 to 1:0.005, still more preferably 1:0.002;
v) diluting in a range from 1:0 a 1:1000, preferably from 1:5 to 1:50, more preferably from 1:5 to 1:30, still more preferably 1:20, the solution obtained in step iv) with at least a polar solvent;
vi) purifying said nanoparticles obtained in steps iv)-v)
(when the dilution is 1:0 the above step ii) is absent);

Glutathione and Li⁺ coated gold nanoparticles wherein said nanoparticle has a diameter from 0.1 to 100 nm.

A method for the preparation of aggregates of LiG-AuNPs comprising the preceding steps (i-iv) of the method, the invention relates to, and an additional step for dispersing said particles in a solvent selected from deionized water, alcohol, hydroalcoholic solution or pharmaceutically acceptable solution or suspension:

Aggregates of nanoparticles as defined in the present description and claims wherein said aggregates have a diameter from 0.1 to 5000 nm.

A pharmaceutical composition comprising aggregates of nanoparticles as defined in the present description and claims in a solvent selected from deionized water, alcohol or hydroalcoholic solution or solution or suspension, and at least a pharmaceutically acceptable excipient and/or carrier.

A kit comprising a plurality of nanoparticles of the invention as defined in the present description and claims and one or more aliquots of deionized water, alcohol or a hydroalcoholic solution or pharmaceutically acceptable solution or suspension.

Gold nanoparticles, or aggregates of nanoparticles, or the composition, or the kit as defined in the present description and claims, for use in a therapeutic treatment.

Additional advantages and/or embodiments of the present invention will be evident from the following detailed description.

### DETAILED DESCRIPTION OF FIGURES

FIG. 1. Microscopic characterization and Dynamic Light Scattering (DLS) of G-AuNPs. (A, B) Representative images acquired in classical Transmission Electron Microscopy of LiG-AuNPs (TEM) with two different magnifications (C-E) Representative images acquired in classical Transmission Electron Microscopy in Scanning mode (STEM) showing images in annular dark field (HAADF, panel C), and the presence of sodium (Na⁺, panel D) and chloride (Cl⁻, panel E) ions in the external crown of the nanoparticle. (F_{a-d}) Analysis in Dynamic Light Scattering (DLS) of LiG-AuNPs left in deionized water at +4° C. for a) 0 days (analysed immediately after ultrasound disaggregation); 7 days; c) 15 days; d) 30 days, showing the presence of: a) dissociated nanoparticles (~3.8 nm with hydrodynamic diameter), b) small aggregates (~250 nm); c) aggregates having average sizes (~300 nm) together with the formation of aggregates with larger sizes; d) big aggregates (>1 μm).

FIG. 2. Internalization of LiG-AuNPs in SH-SY5Y human neuroblastoma cells. (A, B) Representative images of SH-SY5Y human neuroblastoma cells, acquired by transmission laser microscopy (λₑₓ: 514 nm), treated with vehicle (deionized water; panel A) or 1 mg/mL LiG-AuNPs for 24 hours. The arrows show the black spots which are gold dense areas which the light did not succeed in crossing.

FIG. 3: Evaluation of the cytotoxic effect of LiG-AuNPs. Graph showing the percentage of vital cells at the various tested LiG-AuNPs concentrations. Extracellular concentrations of aggregates of LiG-AuNPs 2 mg/mL induce reduction in the cell viability in VERO and SH-SY5Y cells. The cytotoxic concentration thereat half of cells die (CC₅₀) was estimated in 4.3-5.0 mg/mL. n.s. not significant difference vs. vehicle (=0 mg/mL).

FIG. 4. Aggregates of LiG-AuNPs induce phosphorylation of GSK-3β on Ser9, even at low lithium concentration: (A) Representative Western blot of pGSK-3β^{S9} in SH-SY5Y neuroblastoma cells treated for 1 or 24 hours with LiCl (6 mM, corresponding to 6 mEq/L lithium) or LiG-AuNPs at 1 mg/mL (3 mEq/L Li⁺) and 0.05 mg/mL (0.15 mEq/L), left to aggregate for 1-7 days in deionized water; veh, shows the treatment with deionized water, as control. GAPDH was used as loading control; (B) Bar graph showing the ratio between the expression of phosphorylated GSK-3β (Ser9) and total GSK-3β, under the various experimental conditions shown in (A). The treatment with NaG-AuNPs was used as control of LiG-AuNPs a 24 h. The bars represent the average of n=5-10 independent experiments for condition. The dots show the values obtained in the single experiments. **p<0.01 vs. veh; ***p<0.001 vs. veh. n.s., not significant difference vs. veh.

FIG. 5. LiG-AuNPs inhibit the HSV-1 infection in mouse cortical astrocytes (by studying the expression of the ICP4 viral protein in immunofluorescence). (A-D) Representative images acquired by confocal microscopy of murine cortical astrocytes infected by HSV-1 (at multiplicity of infection equal to 1) and immunoprocessed for the ICP4 viral protein (green) 24 hours post-infection. Glial Fibrillary Acidic Protein (GFAP, red) was used to colour the astrocytes. The cellular cores were coloured in blue by staining with DAPI. The panel (A) shows the falsely (mock) infected cells. In panel B the cells infected by HSV-1 without additional treatments are represented. The increase in the red colour (GFAP) shows the presence of astrocytes. In the panels C and D the infected cells were treated respectively with LiG-AuNPs and NaG-AuNPs (1 mg/mL) for the whole post-infection period. (E) Bar graph which quantifies the percentage of ICP4-positive (infected) cells under the conditions represented in the panels A-D. The LiCl condition (6 mEq/L lithium) was added as additional control. A significant reduction in the percentage of the expression of the ICP4 protein expression is observed only in the cells treated with LiG-AuNPs and LiCl. **p<0.001. n.s.: statistically not significant difference.

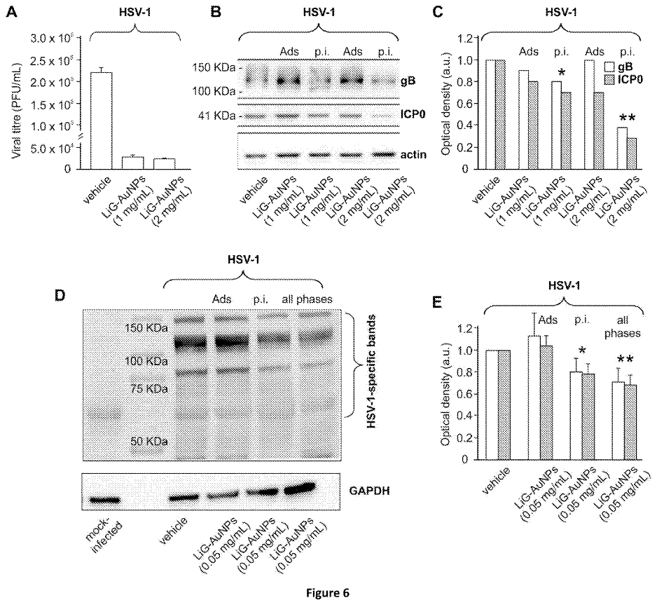

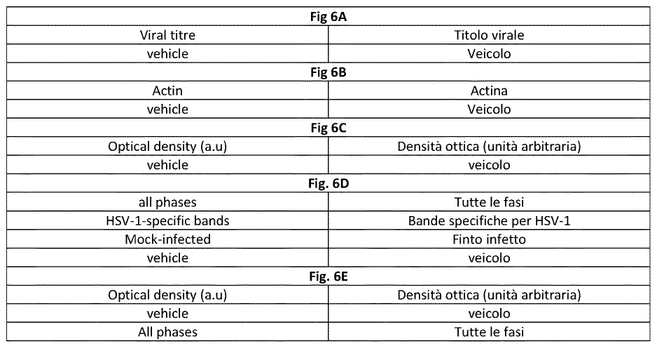

FIG. 6. LiG-AuNPs inhibit HSV-1 infection (by means of the method of plaques and molecular biology/Western Blot). (A) Quantification of the viral titre in terms of plaque-forming units (PLU)/mL by standard assay of the plaques in the supernatants of VERO cells infected by HSV-1 (1 MOI), in absence (vehicle) and presence of LiG-AuNPs (1 and 2 mg/mL), and evaluated 24 hours post-infection. (B) Analysis in Western blot of lysates obtained from VERO cells infected by HSV-1 (1 MOI) treated with vehicle, or LiG-AuNPs (1 and 2 mg/mL) during the adsorption (ADS), or post adsorption (p.i.) period, and analysed 24 hours post infection. The infection is quantified in terms of immunoreactivity for the glycoprotein of the viral envelope gB, or the early viral protein ICPO. Actin is used as loading internal control. (C) Bar graph which quantifies the optical density of the bands represented in panel B. The white bars relate to gB whereas the grey ones to ICP. (D) Representative analysis in Western blot of lysates of SH-SY5Y human neuroblastoma cells infected by HSV-1 (1 MOI) and treated with vehicle (bi-distilled water) or LiG-AuNPs with low concentration (0.05 mg/mL, corresponding to 0.15 mEq/L lithium) during the adsorption period (Ads), the whole post-infection period (p.i) or during all phases of infection (all phases: Ads+ p.i.), and evaluated at the end of the 24 hours p.i. The lysates were probed with a direct antibody against various proteins of HSV-1 virus (pan HSV-1). GAPDH was used as loading control. (E) Bar graph showing the quantification of the bands represented in panel D, HSV-1 (white bars) and ICPO (grey bars, WB not shown). *p<0.05 and **p<0.001 vs. vehicle.

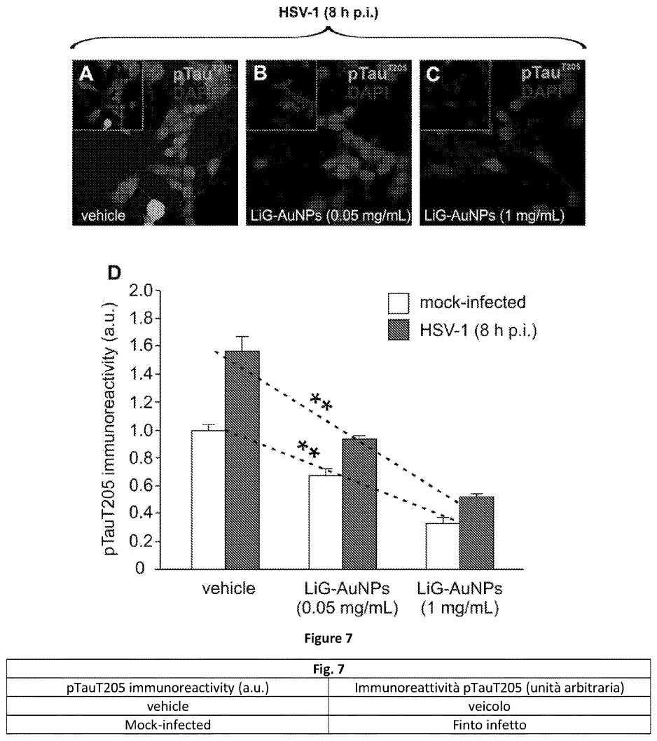

FIG. 7. The treatment with LiG-AuNPs reduces the tau phosphorylation on Thr205 in the cells infected by HSV-1. (A-C) Representative images acquired in confocal microscopy of falsely infected SH-SY5Y cells or cells infected by HSV-1 (1 MOI), and treated with vehicle (A), or LiG-AuNPs at 0.05 mg/mL (B) or 1 mg/mL (C), and fixed 8 hours post-infection and then immunoprocessed for pTau^{T205}. (D) Bar graph which quantifies the immunoreactivity for pTau^{T205} in SH-SY5Y cells under above-mentioned conditions and represented in the panels A-C. The white bars represent the falsely infected (mock) cells or cells infected by HSV-1 (coloured). **p<0.001 for the analysis of linear regression.

FIG. 8. LiG-AuNPs exert an antioxidant action. (A-C) Representative images acquired in confocal microscopy of SH-SY5Y human neuroblastoma cells treated for 24 hours with lipopolysaccharide (LPS, 5 μg/mL) and treated with vehicle (deionized water) or LiG-AuNPs (1 mg/mL), then fixed with PFA (4%) and treated with the fluorescent indicator for the superoxides, dihydroethidium (DHE). (D) Bar graph showing the quantification of the experiments represented in the panels A-C. (E) Bar graph which quantifies the levels of NADPH Oxidase 4 (NOX4) enzyme evaluated by analysis in Western blot (not shown) in VERO cells infected by HSV-1 (1 MOI) and treated or not treated with LiG-AuNPs (1 mg/mL). (F) Representative analysis in Western blot of lysates of VERO cells infected or not infected (mock-infected) by HSV-1 (1 MOI) and then treated with vehicle or LiG-AuNPs (1 or 2 mg/mL, only during the adsorption period of the virus—Ads—or during the whole post-infection time—p.i.—) and probed with direct antibodies against the immature/inactive form of pro-inflammatory cytokine IL-1β (pro-IL-1β, 31 KDa) or against the active (cut) form of the same (IL-1β, 17 KDa). Actin was used as loading internal control. (G) Bar graph showing the quantification of the experiments represented in panel F. The white bars refer to pro-IL-1β, whereas the grey ones refer to IL-1β. **p<0001 ***p<0.0001.

FIG. 9. The intranasal administration of LiG-AuNPs induces phosphorylation of GSK-3β on Ser9 in the hippocampus of mice. (A) Analysis in Western blot for pGSK-3β^{Ser9} and GSK-3 (total) of lysates of hippocampus from C57Bl/6 mice treated for 5 consecutive days with vehicle (left bands) or LiG-AuNPs (at 1, 10 and 100 mg/mL) administered intranasally (3 μL/nostril; bilaterally), and sacrificed 6 hours after the last dose. GAPDH was used as loading internal control. (B) Bar graph which quantifies the values of optical density for pGSK-3β^{Se9} (light grey), GSK-3β “total” (dark grey), and their ratio (red bar/very dark grey), of the analysis WB represented in panel A. The dotted line represents the average level of the value of the controls (vehicle: 0 mg/mL). (C-D) Bar graph which quantifies the values of optical density for pGSK-3β^{Ser9} (light grey), GSK-3β “total” (dark grey), and their ratio (green bar/very dark grey) in lysates extracted from cortex (panel C) and olfactory bulbs (panel D) of the mice treated as in A. *p<0.05; **p<0.01 ***p<0.001; n.s.: not significant

FIG. 10. The intranasal administration of LiG-AuNPs does not induce glial response in the hippocampus of treated mice. (A) Analysis in Western blot for GFAP of lysates of hippocampus from C57B/6 mice treated for 5 consecutive days with vehicle (left bands) or LiG-AuNPs (at 1, 10 and 100 mg/mL) administered intranasally (3 μL/nostril; bilaterally), and sacrificed 6 hours after the last dose. GAPDH was used as loading internal control. (B) Bar graph which quantifies the values of optical density for GFAP. n.s. not significant

FIG. 11. Representative scheme of the operation of LiG-AuNPs. 1) When they are anhydrous, the nanoparticles are separated from each other; 2) when they are put in deionized aqueous environment, the LiG-AuNPs tend to form aggregates in time-depending manner (see FIG. 1); 3) when the aggregates are put in a solution containing monovalent cations (for ex.: Na⁺, K⁺) such as for example culture media for cells, the lithium present on the more external portion of the aggregates is released by exchange of cations, but the most “internal” lithium ions remain protected from the external environment and they cannot be released. 4) when the aggregates of LiG-AuNPs enter the cells, they are disaggregated in single nanoparticles (see Buonerba et al., Scientific Reports 2020), thus the attached lithium is released due to the usual mechanism of cation(s) exchange. The breaking-down of LiG-AuNPs at intracellular level determines an increase in the concentration of Li⁺ ions in the cytosol and/or in the intracellular organelles, thus contributing effectively in increasing the levels of intracellular lithium and in performing its therapeutic action. The lithium so released in the cytosol has the possibility of interacting directly with GSK-3 determining is inhibition and then for all the downstream effects which this entails.

Additional advantages and/or embodiments of the present invention will be evident from the following details description.

### Glossary

LiG-AuNPs (LithiumGlutathione-Gold Nanoparticles), in the present invention relates to gold spherical nanoparticles coated with glutathione functionalized with Lithium(I) ions (that is wherein several functional groups of glutathione are bound to Li⁺ ion) having diameter ranging between 0.1 and 10 nm more probably based upon the developed synthetic procedure, herein claimed, with average diameter of 2 nm.

Aggregates of LiG-AuNPs, according to the present description are aggregates of the above-mentioned nanoparticles obtainable by dispersion for 1-7 days, preferably 7 days of the same in water, alcohol or hydroalcoholic solutions or pharmaceutically acceptable solution or suspension, such aggregates have a diameter of about 100-300 nm and are stable over time at a temperature ranging between +4 and +43° C. for a period of time of 25-35 days to form then aggregates having a diameter of about 1000 nm.

Gold precursor is an inorganic or organic compound of gold and even gold preformed nanoparticles. The gold compound of election for the synthesis of LiG-AuNPs is the tetrachloroauric acid in anhydrous (HAuCl₄; number CAS: 16903-35-8) or trihydrated (HAuCl₄·3H₂O; number CAS: 16961-25-4) form.

Reduced glutathione in the present invention relates to the tripeptide having the formula below and more precisely with number CAS: 70-18-8.

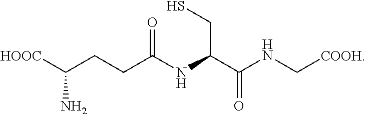 [SMILES: N[C@@H](CCC(=O)N[C@@H](CS)C(=O)NCC(=O)O)C(=O)O]

Basic lithium compound in the present invention relates to any inorganic or organic or metallic compound of lithium capable of lithiating glutathione, that is chemically replacing the carboxylic acid proton with a Li⁺ cation. The anhydrous lithium hydroxide (number CAS: 1310-65-2) or in hydrated from (number CAS: 1310-66-3), is the lithium basic compound of election for the synthesis of LiG-AuNPs.

Lithium salt is an inorganic or organic ionic compound of lithium. Its function is that of easing the formation of aggregates of particles and the precipitation itself of the particles in possible hydroalcoholic solution. The lithium chloride (number CAS: 7447-41-8) is the lithium salt of election for the synthesis of LiG-AuNPs.

Reducing agent of a gold precursor is any chemical compound with lower reducing potential under standard condition than that of the gold under the same conditions.

Sodium borohydride, lithium borohydride, lithium aluminium hydride are some examples of compounds usable for this purpose in order to obtain LiG-AuNPs.

### Solution Suitable to the Administration

The solutions described in the present invention can be in pharmaceutically acceptable form and then suitable for the administration to a patient requiring it.

GSK-3 in the present description has the meaning commonly accepted in the scientific literature and designates Glycogen Synthase Kinase 3, which exists in 2 isoforms (α and β) equally present, although β is the one more associated to degenerative type disorders.

The adjustment of the kinase activity depends upon the phosphorylation site, which is different depending upon the isoform: Ser21 for isoform α; Ser9 for isoform β. pGSK-3 means that the protein GSK-3 is phosphorylated, in superscript after the protein name, and the considered isoform (α/β), one or more aminoacids of the same can be designated which are phosphorylated, for example pGSK-3β^{Ser9} designates that serin in position 9 of protein (isoform β) is phosphorylated, pGSK-3α^{Ser21} that phosphorylation is born by serin in position 21 of isoform α.

It is to be noted that the human protein and the murine one share the same adjustment mechanism. Hereinafter, the swissprot codes of both proteins:
P49841 (GSK3B_HUMAN)
P49840 (GSK3A_HUMAN)
Q9WV60 (GSK3B_MOUSE)
Q2NL51 (GSK3A_MOUSE)

In any part of the description and claims the expression “glutathione and Li⁺ coated gold nanoparticles” can be replaced by any one of the following expressions: “gold nanoparticles coated with lithium salt of glutathione”; “gold nanoparticles coated with glutathione and lithium cations” or “gold nanoparticles coated with glutathione and Li(I)”. In any part of the description and claims the term “comprising” can be replaced by “consisting of”.

The term Li(I) in the present description, as well as in the chemical nomenclature, is a synonym of the term Li⁺.

### DETAILED DESCRIPTION

The present invention relates to a method for the preparation of glutathione and Li⁺ coated gold nanoparticles, comprising the following steps:
i) preparing a mixture of
   a gold precursor in a concentration from 0.0001M a 10M, preferably da 0.001M a 0.1M, more preferably from 0.010M to 0.020M, still more preferably 0.017M;
   at least a polar solvent;
   glutathione in a molar ratio with respect to said gold precursor from 0.1:1 to 100:1, preferably 1:1 to 1:0.1;
   a basic lithium compound in a molar ratio with respect to said gold precursor from 0.01:10 to 10:0.01, preferably 1:1 to 1:0.1, more preferably from 1:0.05 to 1:0.15, still more preferably 1:0.09;
at a temperature ranging from −20° to 120° C., preferably at room temperature, in an environment equipped with a stirring and shaking system, to obtain a clear colourless solution;
ii) diluting in a range from 1:0 a 1:1000, preferably from 1:5 to 1:50, more preferably from 1:5 to 1:30, still more preferably 1:20, the solution obtained in step i) with at least a polar solvent;
iii) adding to the diluted solution of step ii) a reducing agent of said gold precursor in a molar ratio with respect to said gold precursor from 1:1 a 100:0.1, preferably 1:1 to 1:0.1, more preferably from 1:0.1 to 1:0.3, still more preferably 1:0.22, to obtain a colloidal suspension of gold nanoparticles, at a temperature ranging from −20° to 120° C., preferably at room temperature, in an environment equipped with a stirring and shaking system;
iv) adding a lithium sale in a molar ratio with respect to said gold precursor from 0.1:1 to 100:0.001, preferably from 1:0.001 to 1:1, more preferably from 1:0.001 to 1:0.005, still more preferably 1:0.002;
v) diluting in a range from 1:0 a 1:1000, preferably from 1:5 to 1:50, more preferably from 1:5 to 1:30, still more preferably 1:20, the solution obtained in step i) with at least a polar solvent;
vi) purifying said nanoparticles obtained in steps iv)-v).

The concentration of gold precursor in step i) of the method according to the present invention can be any punctual value until the third digit after the comma of concentration from 0.0001M to 10M, in a preferred form said ratio is from 0.010M to 0.0030M, preferably from 0.010M to 0.020M, still more preferably 0.011M, 0.012M, 0.013M, 0.014M, 0.015M, 0.016M, 0.017M, 0.018M or 0.019M.

The molar ratio of glutathione with respect to the gold precursor in step i) of the method of the present invention can be any ratio present in the above-said range from 0.1:1 to 100:0.01, in an embodiment said ratio is preferably from 1:0.6 a 1:0.3, still more preferably 1:0.55; 1:0.50; 1:0.45, 1:0.40 or 1:0.35.

The molar ratio of the basic lithium compound with respect to the gold precursor in step i) of the method of the present invention can be any ratio present in the above-said range from 0.01:10 to 10:0.01, in an embodiment said ratio is preferably from 1:0.15 to 1:0.05, still more preferably 1:0.14; 1:0.13; 1:0.12, 1:0.11, 1:0.10, 1:0.09; 1:0.08; 1:0.07 or 1:0.06.

In a preferred embodiment, in step i) of the method according to the present invention, the concentration of gold precursor is selected from 0.010M a 0.020M, still more preferably 0.011M, 0.012M, 0.013M, 0.014M, 0.015M, 0.016M, 0.017M, 0.018M or 0.019M; the molar ratio of glutathione with respect to the gold precursor is selected from 1:0.55; 1:0.50; 1:0.45, 1:0.40 or 1:0.35 and the molar ratio of the basic lithium compound with respect to the gold precursor is selected from 1:0.14; 1:0.13; 1:0.12, 1:0.11, 1:0.10, 1:0.09; 1:0.08; 1:0.07 or 1:0.06.

In a still more preferred embodiment, in step i) of the method according to the present invention, the concentration of gold precursor is between 0.010M and 0.020M, in particular 0.017M; the molar ratio of glutathione with respect to the gold precursor is 1:0.45 and the molar ratio of the basic lithium compound with respect to the gold precursor 1:0.09. The temperature in step i) of the method according to any embodiment of the present invention as described herein is preferably a temperature between 10° C. and 50° C., still more preferably room temperature (conventionally the room temperature is considered a temperature of approximately 25° C.).

The dilution in steps ii) and v) of the method of the present invention is any dilution from 1:0 to 1:1000 of the obtained dilution, respectively, in step i) and in step iv) with at least a polar solvent, preferably said dilution is a dilution from 1:5 to 1:30, still more preferably is a dilution 1:15, 1:16, 1:17, 1:18, 1:19, 1:20, 1:21, 1:22, 1:23, 1:24 or 1:25. In a most preferred embodiment said dilution is 1:20. When the dilution is 1:0, it is clear that one or more dilution steps of the method can be omitted.

In a preferred embodiment, in step i) of the method according to the present invention, the concentration of gold precursor is 0.017M; the molar ratio of glutathione with respect to the gold precursor is 1:0.45 and the molar ratio of the basic lithium compound with respect to the gold precursor 1:0.09, the temperature is room temperature and the dilution in step ii) is a dilution 1:20.

According to the present invention the molar ratio between the reducing agent and the gold precursor in step iii) of the method is any ratio from 1:1 to 100:0.10. Preferably said ratio is any ratio from 1:0.30 to 1:0.10, still more preferably said ratio is 1:0.25; 1:0.24; 1:0.23; 1:0.22; 1:0.21 o 1:0.20. In a still more preferred embodiment said ratio is 1:0.22.

In a preferred embodiment, in step i) of the method according to the present invention, the concentration of gold precursor is 0.017M; the molar ratio of glutathione with respect to the gold precursor is 1:0.45 and the molar ratio of the basic lithium compound with respect to the gold precursor 1:0.09, the temperature is room temperature, the dilution in step ii) is a dilution 1:20 and the molar ratio between the reducing agent and the gold precursor in step iii) is 1:0.22.

The temperature in step iii) of the method according to any embodiment of the present invention as described herein is preferably a temperature between −20° C. and 120° C., still more preferably room temperature (conventionally the room temperature is considered a temperature of approximately 25° C.).

In a preferred embodiment, in step i) of the method according to the present invention, the concentration of gold precursor is 0.017M; the molar ratio of glutathione with respect to the gold precursor is 1:0.45 and the molar ratio of the basic lithium compound with respect to the gold precursor 1:0.09, the dilution in step ii) is a dilution 1:20, the molar ratio between the reducing agent and the gold precursor in step iii) is 1:0.22 and the temperature in steps i) and iii) is room temperature.

According to the invention, the lithium salt added in step iv) of the method is in any molar ratio from 0.1:1 to 100:0.001 with respect to the gold precursor, preferably from 1.005 to 1:0.001; still more preferably said ratio is 1:0.004; 1:0.003 or 1:0.002. In a preferred embodiment said ratio is 1:0.002.

In a preferred embodiment, in step i) of the method according to the present invention, the concentration of gold precursor is 0.017M, the molar ratio of glutathione with respect to the gold precursor is 1:0.45 and the molar ratio of the basic lithium compound with respect to the gold precursor 1:0.09, the molar ratio between the reducing agent and the gold precursor in step iii) is 1:0.22, the temperature in steps i) and iii) is room temperature, the ratio between the lithium salt and the precursor in step iv) is 1:0.002 the dilution in step ii) is a dilution 1:20.

According to the present invention, when the dilution ratio is 1:0 in steps ii) and/or v), it is evident that no dilution is performed. Therefore, the method of the invention also comprises an embodiment wherein the dilution steps ii) and/or v) are not performed. In such embodiment, the method of the present invention is represented by the following steps:
i) preparing a mixture of
   a gold precursor in a concentration from 0.0001M to 10M, preferably from 0.001M to 0.1M, more preferably from 0.010M to 0.020M, still more preferably 0.017M;
   at least a polar solvent;
   glutathione in a molar ratio with respect to said gold precursor from 0.1:1 to 100:0.1, preferably 1:1 to 1:0.1;
   a basic lithium compound in a molar ratio with respect to said gold precursor from 0.01:10 to 10:0.01, preferably 1:1 to 1:0.1, more preferably from 1:0.05 to 1:0.15, still more preferably 1:0.09;
at a temperature from −20° C. to +120° C., preferably at room temperature, in an environment equipped with a stirring and shaking system, to obtain a clear colourless solution;
OPTIONAL [ii) diluting in a range from 1:0 to 1:1000, preferably from 1:5 to 1:50, more preferably from 1:5 to 1:30, still more preferably 1:20, the solution obtained in step i) with at least a polar solvent]
iii) adding to the diluted solution of step ii) a reducing agent of said gold precursor in a molar ratio with respect to said gold precursor from 1:1 to 100:0.1, preferably 1:1 to 1:0.1, more preferably from 1:0.1 to 1:0.3, still more preferably 1:0.22, to obtain a colloidal suspension of gold nanoparticles, at a temperature from −20° C. a +120° C., preferably at room temperature, in an environment equipped with a stirring and shaking system;
iv) adding a lithium salt in a molar ratio with respect to said gold precursor from 0.1:1 to 100:0.001, preferably from 1:0.001 to 1:1, more preferably from 1:0.001 to 1:0.005, still more preferably 1:0.002;
OPTIONAL [v) diluting in a range from 1:0 to 1:1000, preferably from 1:5 to 1:50, more preferably from 1:5 to 1:30, still more preferably 1:20, the solution obtained in step i) with at least a polar solvent]
vi) purifying said nanoparticles obtained in steps iv)-v).

It is clear then that step ii) and/or step v) of dilution are optional steps of the method of the present invention.

According to the present invention, said gold precursor is selected from gold halides, gold chalcogens, gold pycnogens, gold crystallogens or gold complexes and clusters (I or III) or organoauric compounds or mixtures thereof. Preferably said gold precursor is the tetrachloroauric acid in trihydrated form.

According to the invention, said polar solvent is selected from an aprotic polar solvent such as for example acetone, acetonitrile, tetrahydrofuran, dioxane, dimethyl sulfoxide, dimethylformamide, peralkylated ureas such as tetramethylurea and 1,3-dimethyl-2-imidazolidinone, and hexamethylphosphoramide, or it can be a protic polar solvent such as an alcohol, a hydroalcoholic solution, a carboxylic acid, an ammine or a sulfonated or nitrated compound or a mixture thereof. Preferably the polar solvent of election for the synthesis of LiG-AuNPs is methanol in case in mixture with one or more additional solvents, preferably a hydroalcoholic solution of methanol (for example a hydroalcoholic solution of methanol from 20% to 25%, to 30%), a mixture comprising water and methanol.

In a still more preferred embodiment said polar solvent is a hydroalcoholic solution of 25% methanol.

In an embodiment, said glutathione is glutathione in reduced form (CAS: 70-18-8).

In an additional embodiment said basic lithium compound is selected from anhydrous or monohydrate lithium hydroxide, lithium oxide, lithium hydride, lithium alkoxides, lithium amides, lithium carbonate, lithium bicarbonate, lithiated Zintl compounds, metallic lithium, lithium/ammonia solutions, lithium amalgam, lithiated anion resins, lithium phosphates, lithium sulfates, lithium carboxylic compounds, lithium tetraborate, lithiated borates, lithium fluoride, lithium hypochlorite, lithium chlorite, lithium oxyanions, organolytic compounds or mixtures thereof. Preferably said basic lithium compound is the lithium hydroxide monohydrate.

In an embodiment, said reducing agent of said gold precursor is selected from sodium borohydride, lithium borohydride, lithium aluminium hydride. Preferably said reducing agent is sodium borohydride.

In an embodiment, said lithium salt is an inorganic or organic ionic compound of lithium preferably selected from lithium chloride, lithium iodide, lithium fluoride, lithium bromide, lithium oxide, lithium hydroxide, lithium sulphide. Preferably said lithium salt is lithium chloride.

In an embodiment of the method of the invention, said gold precursor is tetrachloroauric acid trihydrate, said polar solvent is a mixture comprising methanol and water, said glutathione is glutathione in reduced form (GSH), said basic lithium compound is lithium hydroxide monohydrate, said reducing agent of the gold precursor is sodium borohydride, and said lithium salt is lithium chloride.

In an embodiment, said step (iv) is performed by stirring for a time ranging from 1 to 120 hours, preferably 48 hours. The constant stirring can be performed by the stirring systems known to the person skilled in the art, such as for example with magnetic anchor and magnetic stirrer.

In a preferred embodiment, said step vi) is carried out by removal of the supernatant liquid by sedimentation, or centrifugation, or dialysis, filtration or centrifugal ultrafiltration, or combinations thereof, and subsequently drying in vacuum or under air or by heating or by gas flow or freeze-drying or combinations thereof. The purification procedures are performed according to the common techniques known to the person skilled in the art.

In particular, the precipitation of the nanoparticles can be obtained in an Imhoff cone.

An additional object of the present invention is represented by a glutathione and Li⁺ coated gold nanoparticle, wherein said coated nanoparticle has a diameter from 0.1 to 100 nm. Preferably, based upon the developed procedure, herein claimed and detailed in example 1, said nanoparticle has a spherical shape and average diameter of 2 nm.

In an embodiment of the nanoparticle of the invention, said gold is present in an amount from 40 to 60% w/w, said glutathione is present in an amount from 20 to 30% w/w and said lithium is present in an amount from 0.1 to 10% w/w.

In an embodiment, said nanoparticle can be obtained by the method the present invention relates to. An object of the invention is then a glutathione and Li⁺ coated gold nanoparticle as described above, which can be obtained by the method of the present invention.

The present invention also relates to a method for the preparation of aggregates of gold nanoparticles defined in the present description, comprising a step of dispersing said particles in a solvent selected from deionized water, alcohol or hydroalcoholic solution or pharmaceutically acceptable solution or suspension (and then suitable to the administration) for a time of at least one minute at a temperature ranging from +4 to 56° C., preferably from 35° C. to 40° C., in such an amount as to obtain a concentration of said nanoparticles from 0.00001 to 1000 mg/mL, preferably from 1 to 100 mg/mL.

In particular, said step of dispersing said particles occurs for a time of at least 1-7 days, preferably at least 7 days, at a concentration of said nanoparticles of 100 mg/mL, at a temperature of +4° C. for forming the aggregates, and is performed by an additional dispersing step (from +4 to 43° C., preferably at 37° C.), in amounts of said nanoparticles so as to obtain a concentration of said nanoparticles from 0.01-1 mg/mL for in vitro applications and 10-100 mg/mL for in vivo applications.

In an embodiment, said nanoparticles are in dry form.

In an embodiment, said solvent is deionized water is at a pH of 5.5 to 6.5, preferably 5.8.

In an embodiment of the method, said dispersion step can be preceded by steps (i-vi) of the method for preparing the nanoparticles the present invention relate to.

The method for preparing aggregates of nanoparticles of the present invention also allows to obtain compositions comprising aggregated nanoparticles.

The present invention also relates to aggregates of gold nanoparticles defined in the present description, wherein said aggregate has a diameter from 0.1 to 5000 nm, preferably between 10 and 300 nm, still more preferably between 100 and 300 nm.

Moreover, such aggregates have a icosahedral morphology.

In an embodiment, said aggregates can be obtained by the method for the preparation of aggregates of gold nanoparticles the present invention relates to.

The present invention also relates to a pharmaceutical composition comprising aggregates of glutathione and Li⁺ coated nanoparticles according to claim 18 or 19, a solvent selected from deionized water, alcohol, hydroalcoholic solution, a pharmaceutically acceptable solvent or a pharmaceutically acceptable suspender and at least a pharmaceutically acceptable excipient and/or carrier. The person skilled in the art knows the possible excipients and vehiculants useful for the purposes of the present invention.

In an additional embodiment, said pharmaceutical composition is in a form suitable for the administration by oral, systemic, parenteral, injection, intravenous, aerosol, nebulization, topical, intranasal, nasopharyngeal and/or oropharyngeal, rectal, intravaginal route and is in the form of a cream, ointment, salve, aerosol, solution, suspension, gel, hydrogel, emulsion, soft or hard gelatine capsule, nebulizable solution or suspension.

In an embodiment the pharmaceutical composition is in two-component form to be mixed before use and it comprises the nanoparticles of the invention in dry form suitably dosed and a suitable solvent in suitable dosage. Or in one single container with separate compartments or in two different containers.

The invention also relates to a kit comprising a plurality of nanoparticles as defined in the present description, and one or more aliquots of a solvent selected from deionized water, alcohol, pharmaceutically acceptable solvent, pharmaceutically acceptable suspender, or hydroalcoholic solution.

At last, the present invention relates to the gold nanoparticles according to the present description, or the aggregates of nanoparticles, or the composition, or the kit, as defined in the present description and claims, for use in a therapeutic treatment.

In an embodiment, the gold nanoparticles, or the aggregates of nanoparticles, or the composition, or the kit, as defined in the present description and claims, are for use in the treatment or as adjuvant in a treatment of infectious diseases, of infections caused by DNA or RNA viruses, neurodegenerative diseases and mood disorders, all related to the activation or positive modulation of Glycogen Synthase Kinase-3 (GSK-3). In other terms infectious, neurodegenerative diseases and mood disorders which take advantage from the inhibition of Glycogen Synthase Kinase-3 (GSK-3).

In particular, wherein said inhibition of GSK-3 is performed by phosphorylation of the amino acid serine in position 9 for isoform β (pGSK-3β^{Ser9}) and of the amino acid serine in position 21 for isoform α (pGSK-3α^{Ser21}) In an embodiment, said infectious, neurodegenerative disease and mood disorders correlated to the activation or positive modulation of the activity of Glycogen Synthase Kinase-3 (GSK-3) are selected from Alzheimer disease, Parkinson disease, Huntington disease, tautopathies such as for example FDT (Frontotemporal dementia), PSP (progressive supranuclear palsy), PART (Primary age-related tauopathy) etcetera.; and said infections caused by DNA or RNA viruses are selected from infections caused by Coronavirus, Orthomyxovirus, Filovirus, Flavivirus, Hepadnavirus, Hepevirus, Herpesvirus, Papillomavirus, Pneumovirus, Poxivirus, Rhinovirus, Reovirus, Togavirus, a flu virus.

In a preferred embodiment, said Herpes virus is Herpes Simplex type 1 (HSV-1), and said Coronavirus is SARS-CoV-1 and SARS-CoV-2.

In an additional embodiment, the aggregates of nanoparticles, or the composition, as defined in the present description and claims, are administered by oral, systemic, parenteral, injection, intravenous, aerosol, nebulization, topical, intranasal, nasopharyngeal and/or oropharyngeal, rectal, intravaginal route in said treatment or said adjuvant of said treatment, at a concentration of said nanoparticles between 1 and 100 mg/mL.

The invention also relates to a therapeutic method for the treatment of diseases as described above, comprising one or more steps, to a subject requiring it, for administering the gold nanoparticles, or the aggregates of nanoparticles, or the composition, as defined in the present description and claims, they are administered by oral, systemic, parenteral, injection, intravenous, aerosol, nebulization, topical, intranasal, nasopharyngeal and/or oropharyngeal, rectal, intravaginal route in said treatment or said adjuvant of said treatment.

In any part of the present description and claims, the term “comprising” can be replaced by the term “consisting of”.

Each embodiment related to a feature of the present description can be combined with one or more of the other embodiments related to different features. In particular, the combinations between the preferred or most preferred embodiments of each feature of the herein described method of the invention, the nanoparticles, the aggregates, the composition or the kit are preferred.

Examples are reported hereinafter which have the purpose of better illustrating the methods disclosed in the present description, such examples are in no way to be considered as a limitation of the preceding description and the subsequent claims.

Hereinafter the declarations required by Art 170 bis CPI about biological matter are inserted.

In compliance with Art. 170bis comma 2 C.P.I and according to Art. 21 paragraph 2 of the Implementation regulation of C.P.I. adopted with M. D. 13.1.2010 n.33, it is declared that:
the material of animal/vegetable origin underlying the invention, the above-mentioned application relates to, has murine and human provenance. As far as the murine provenance is concerned, the cells are astrocytes and/or primary neurons, obtained by C57Bl/6 mouse (Ministry of Health Authorization: Nr. 594/2022-PR). As far as the cells of human derivation are concerned, they are tumour cells (neuroblastoma SH-SY5Y, carcinoma of the uterine cervix HeLa, epithelial cells of monkey kidney (VERO), etc., purchased by the company ATCC.

In compliance with Art. 170bis comma 4 C.P.I, it is declared that:
with reference to the biological material, containing microorganisms or genetically modified organisms, object or used in the present application the obligations deriving from national or community regulations, and in particular, by the provisions referred to in paragraph 6 of I.d. of 12 Avril 2001 n. 206 and 8 Jul. 2003 n. 224, relating such modifications, were respected.

### EXAMPLES

The nanoparticles the present invention relates to, LiG-AuNPs, were synthetized by modification of a literature procedure, already illustrated by some of the proponents of the current invention for the analogous NaG-AuNPs systems, described in Buonerba et al., Scientific Reports, 2020, 10: 11380. By way of not-exhaustive and not limiting example, hereinafter the synthesis and the applications of LiG-AuNPs are reported.

The average elementary composition of LiG-AuNPs results to be: Li=2.0±0.4%, C=17.3±0.2%, N=5.4±0.1%, S=4.4±0.2% and Au=53.6±0.3%, corresponding approximately to a molar ratio Li/C/N/S/Au=1,1/5,3/1,4/0,5/1 wherein all carboxylic functionalities of glutathione are lithiated. The analysis with transmission electron microscope (TEM) of LiG-AuNPs highlights an icosahedral morphology of the gold clusters with diameter ranging from 0.5 and 5 nm, more precisely of 2 nm (FIG. 1A,B).

When re-dispersed in deionized water, LiG-AuNPs form quickly (few seconds) aggregates with diameter comprised in the range of 10-300 nm of diameter, characterized by analysis of dynamic light scattering (DLS). More precisely, when dispersed in deionized water (pH: 5.8, T=37° C.) at concentrations comprised between 0.01 and 100 mg/mL the formation of aggregates with average diameter centred in the range 250-300 nm is observed. Such aggregates are stable over time until 30 gg in deionized water. Within such time period even the formation of aggregates with larger diameter is observed (FIG. 1F).

Determination of the lithium release: Buonerba et al., Scientific Reports 2020 shows the TEM micrography of hepatocarcinoma tumour cells (HepG2) incubated for 1 hour in culture medium comprising a 0.07-0.7 mg/mL concentration of aggregates NaG-AuNPs. The internalization of these aggregates was observed, followed by their decomposition in the cytosol and/or in the intracellular organelles in single AuNPs. FIG. 2 shows the internalization of LiG-AuNPs in cells of human neuroblastoma SH-SY5Y incubated for 24 h with such particles at the concentration of 1 mg/mL. Considering the morphological and compositional analogy of NaG-AuNPs and LiG-AuNPs it is possible to hypothesize a similar behaviour in the internalization process of the latter, as otherwise demonstrated in the subsequent paragraphs of the present document. The release of Li⁺ by LiG-AuNPs in cellular environment, was studied, by way of not exhaustive example, in culture medium Dulbecco's Modified Eagle Medium (DMEM; Dulbecco et al., 1959), containing a high saline concentration particularly enriched with sodium, suitable to mime the extra-cellular environment. The LiG-AuNPs, dispersed in DMEM previously purified by centrifugal ultrafiltration, tend to release Li⁺ ions, mainly by Li⁺/Na⁺ exchange, by making these cations available for their extracellular pharmacological action. The nanoparticles were dispersed in the medium by sonication for 5 min then removed after 1 and 24 hours by filtering small columns (cutoff 5 KDa) and then analysed by optical emission spectroscopy of inductively coupled plasma (ICP-OES). A lithium content of 0.0017±0.0001 mg/L, 16.71±0.36 mg/L and 18.28±0.08 mg/L was found respectively per pure MEM, and MEM after 1 hour and 24 hours of exposure to LiG-AuNPs. Then, monodispersed nanoparticles released 83.5 and 91.4% of lithium in MEM after 1 and 24 h. The residual Li⁺ cations (between 10 and 15%) remained adhered electrostatically to the internalized LiG-AuNPs and allow a significant accumulation of intracellular lithium.

Determination of the use optimum concentration: the use limit concentration of LiG-AuNPs was preliminarily evaluated by measurements of cell viability 90% by using the exclusion test with Trypan blue on epithelial cells of monkey kidney (VERO cells) and SH-SY5Y human neuroblastoma cells treated for 24 hours with LiG-AuNPs at increasing concentrations (range 0.1-10.0 mg/mL). A limit concentration for using LiG-AuNPs was determined equal to 2.0 mg/mL (corresponding to 6 mEq/L extracellular Li⁺), within which the cell viability keeps higher than 90%, with a cytotoxic concentration (CC)₅₀ equal to 4.3-5.0 mg/mL (FIG. 3).

Determination of the lithium intracellular internalization effectiveness (uptake) by LiG-AuNPs: The cellular uptake of Li⁺ after extracellular treatment with LiG-AuNPs was determined by ICP-OES spectroscopic analysis of the cellular lysates digested in acids, and compared with a same treatment with lithium chloride (LiCl). SH-SY5Y human neuroblastoma cells in culture were subjected to a treatment of 24 hours with 3 mM LiCl (corresponding to 3 mEq/L extracellular Li⁺) and 1 mg/mL LiG-AuNPs (corresponding to 3 mEq/L extracellular Li⁺). At the end of the treatment, the intracellular concentration of Li⁺ in the cells treated with LiG-AuNPs results to be higher by about 26 times (range: 9×-44×) than that of the cells treated with LiCl, with values of 0.154-0.3375 pg/cell in the treatment with LiG-AuNPs and 0.007-0.0375 pg/cell with LiCl.

Considering that the inhibition of GSK-3 is typically obtained in the in vitro experiments with concentrations of extracellular LiCl in the range 5-20 mM (with a IC50 of 1 mM), the LiG-AuNPs allow to obtain the same effective intracellular concentration of Li⁺ at significantly lower extracellular concentration levels.

Determination of effectiveness of LiG-AuNPs on the inhibitory phosphorylation of GSK-3β: The action effectiveness of LiG-AuNPs on the intracellular molecular targets was tested by evaluating the inhibitory phosphorylation of GSK-3β (on Ser 9) in various experimental paradigms suitable to determine both the effective concentration thereof and the transversality of the effects. In all hereinafter described experiments, LiG-AuNPs were used suspended in double-distilled H₂O for about 7 days at the stock concentration of 100 mg/mL.

### In Vitro Studies

Human neuroblastoma cells (SH-SY5Y) were incubated for 1 hour and 24 hours with: J) vehicle (culture medium DMEM/Ham's F12); ii) aggregates (^{~}250 μm) of LiG-AuNPs at the concentration of 1 mg/mL (equal to 3 mEq/L of extracellular lithium); iii) LiCl 6 mM (equal to 6 mEq/L of extracellular lithium), concentration usually used for in vitro studies for the inhibition of GSK-3 (Zhang et al., 2003). The phosphorylation of GSK-3β on Serina 9 (pGSK-3β^{S9}) was evaluated by experiments of Western Blot (WB) on intracellular lysates of the treated cells, with respect to the expression of the total protein. By placing equal to “1” the ration between the expression of pGSK-3β^{S9} and that of total GSK-3β (pGSK-3β^{S9}/GSK-3β=1) under “vehicle” condition, the following values were obtained under the remaining experimental conditions (FIG. 4):
i) for treatment of 1 h: 1 mg/mL LiG-AuNPs: 1.60±0.20 (n=4 independent experiments; p=0.048 vs. vehicle); 6 mM LiCl: 1.78±0.25 (n=4; p=0.019 vs. vehicle; not significant vs. 1 mg/mL LiG-AuNPs).
ii) for treatment of 24 h: 1 mg/mL LiG-AuNPs: 2.23±0.26 (n=10 independent experiments; p<1×10⁻³ vs. vehicle); 6 mM LiCl: 2.45±0.29 (n=10; p<1×10⁻³ vs. vehicle; not significant—p=0.982—vs. 1 mg/mL LiG-AuNPs);

The statistical significance was evaluated by ANOVA test followed by Tukey post-hoc correction.

In order to demonstrate the effectiveness of treatment with LiG-AuNPs in inducing inhibitory phosphorylation (Ser9) of GSK-3β with respect to the lithium salts, the SH-SY5Y cells were treated for 1 and 24 hours with low concentrations of LiG-AuNPs (0.05 mg/mL, corresponding to 0.15 mEq/L extracellular Li⁺) and an analogous concentration of LiCl. Under these conditions, LiG-AuNPs are still capable of determining significant increases in the pGSK-3β^{Ser9}/GSK-3β ratio [1.47±0.13 (n=4) after 1 h and 1.54±0.12 (n=8) after 24 h; p=0.023 and p<1×10⁻⁴ vs. vehicle=1, respectively). On the contrary, the LiCl at the concentration of 0.15 mM (equal to 0.15 mEq/L) did not induce any significant modification after 24 hours (pGSK-3ρ^{Ser9}/GSK-3β=1.14±0.14; n=4; not significant—p=0.495—vs. vehicle). The LiCl condition at low dose (0.15 mM) for 1 hour was not tested considering the not significant effect after 24 hours of treatment.

Based upon their capability of internalization in cells, NaG-AuNPs (1 mg/mL for 24 h) were used as control of LiG-AuNPs for the phosphorylation of GSK-3β on Ser9 (FIG. 4B). As expected, the NaG-AuNPs did not determine any significant modification of pGSK-3β^{S9} (1.32±0.29, n=4; p=0.794 vs. vehicle=1; FIG. 4B; right black column).

At last, the transversatility of the effect of LiG-AuNPs in other cellular models was confirmed by evaluating pGSK-3β^{S9} in cortical astrocytes of mouse or human lung carcinoma cells (A549) treated for 24 hours with 1 mg/mL LiG-AuNPs, by obtaining overlapping results.

Such data demonstrate that the LiG-AuNPs are more effective than LiCl in inducing inhibitory phosphorylation of GSK-3,6 under all tested conditions, even at very low doses of extracellular lithium (0.15 mEq/L) when LiCl is not capable of exerting any significant effect.

The effectiveness of the lithiated nanoparticles was further tested even on phosphorylation of tau protein in a model of increased phosphorylation, such as the infection by HSV-1 (De Chiara G et al., Plos Pathogens, 2019). As shown in FIG. 7, the LiG-AuNPs applied for 24 hours (0.05 mg/L-0.15 mEq/L Li⁺) reduce significantly the immunoreactivity of cells for the phosphorylated tau protein on threonine 205, that is under both infection and control condition.

### In Vivo Studies

The effectiveness of LiG-AuNPs in determining the modulation of GSK-3β at brain level was tested by intranasal administration in a murine animal model, as alternative method to the administration of lithium by systemic route. Generally, the intranasal administration represents the best selection for a “drug-delivery” in the brain, considering that it provides a not invasive method to bypass the blood brain barrier [Hanson and Frey, 2008]. The LiG-AuNPs were administered for consecutive 5 days, 3 μL per nostril, at the concentrations of 1 mg/mL (3 mEq/L of lithium(I)) 10 and 100 (300 mEq/L of lithium (I)) in double-distilled H₂O in C57Bl/6 mice, and the animals were sacrificed 6 hours after the last administration with the purpose of removing the brain.

The selection of the concentrations for the mouse was based upon in vitro experiments, and upon information deriving from the normal doses of lithium usually used in human patients. In fact, the commercially available tablets of lithium carbonate (Li₂CO₃) contain 300 mg of lithium salt, corresponding to about 0.018 mg lithium/gram of body weight.

Considering that the LiG-AuNPs contain about 2% of lithium, the maximum concentration which was tested (6 μL at 100 mg/mL×5 days) corresponds to about 0.012 mg lithium/gram of body weight (for a mouse), then in line with the dose for human beings.

For each removed brain, a hemisphere was analysed in toto by ICP-OES after lyophilization for determining the gold concentrations, whereas the other hemisphere was used for determining pGSK-3β^{Ser9}, GSK-3β and their ratio, in various brain areas such as hippocampus, cortex and olfactory bulbs, by measurements of WB. The performed analyses demonstrated that the intranasal administration of LiG-AuNPs was capable of modulating GSK-3β in brain (FIG. 9), and in particular at the hippocampus level. In such area increased levels of pGSK-3β^{Ser9} were found with respect to the total protein (GSK-3β) in the mice treated with LiG-AuNPs with respect to those treated with the vehicle. In fact, by placing equal to 1.00±0.06 the value of ratio pGSK-3β^{Ser9}/GSK-3β in the vehicle, the other values were: 1.67±0.27 (1 mg/mL, n=7; p=0.083 vs. vehicle), 2.02±0.45 (10 mg/mL, n=9; p=0.016 vs. vehicle), and 2.37±0.62 (100 mg/mL, n=9; p=0.006 vs. vehicle) (FIG. 9A,B). Significant modifications at the level of the cortex and olfactory bulbs were not observed (FIG. 9C,D).

ICP-OES spectroscopy detected the presence of gold in all analysed hemispheres, with an average value of 72.8±18.6 and 84.2±6.8 ng/hemisphere under the condition of 10 and 100 mg/mL of LiG-AuNPs, respectively, showing that the AuNPs reached the brain.

At the concentration of 1 mg/mL, ICP-OES spectroscopy is not capable of detecting the presence of gold probably because it is below the detection threshold of the used instrument.

Such studies, besides, show 10 mg/mL of LiG-AuNPs as the optimum dose of intranasal administration for the mice.

### Determination of the Effectiveness of LiG-AuNPs in Reducing the Production of Superoxides

In LiG-AuNPs, the lithium ions are attached covalently to the gold by the sulphur atoms present in glutathione (GSH). Besides, it is known that GSH exerts an important antioxidant action by the sulphur (Mukwevho et al., Molecules. 2014). Let's assume then that the LiG-AuNPs could perform an antioxidant action for supporting the beneficial action of lithium. The antioxidant action of LiG-AuNPs (1 mg/mL) on SH-SY5Y human neuroblastoma cells treated for 24 hours with lipopolysaccharide (LPS, 5 μg/mL), known as inducing oxidative stress on cells, was tested. At the end of the treatment with LPS, the cells were incubated with the fluorescent probe for the superoxides, designated “dihydroethidium” (DHE). Our results show that the treatment of cells with LPS induces an increase in fluorescence intensity equal to 28% (p=1.4×10⁻³³ vs vehicle). The treatment with LiG-AuNPs brings fluorescence back to the control values (p=7.6×10⁻³³ vs. cells treated with LPS; FIG. 8A-D) showing the antioxidant action of LiG-AuNPs. As additional confirmation, in the cells infected by HSV-1, the enzyme NADPH Oxidase 4 (NOX4), known as participating in the production of the reactive species of oxygen (ROS) and of pro-oxidant interleukin 1β, both immature/inactive (pro-IL-1β, 31 KDa) and mature/active (IL-1β, 17 KDa), were quantified.

Through experiments of Western blot, we verified that the treatment with LiG-AuNPs performed in the 24 post-infection hours, reduced significantly the levels of NOX4 (−50%; FIG. 8E) as well as the levels of IL-1β (FIG. 8F,G).

### Determination of Effectiveness of LiG-AuNPs on the Targets Downstream of GSK-3β (Infection by Virus Herpes Simplex Type 1 and Phosphorylation of Tau Protein)

It is known that GSK-3β plays a key role in the viral infections, both for RNA virus and DNA virus. Among the various viruses which use GSK-3β to infect the cells there is Herpes Simplex type 1 (HSV-1). The research group of one of the inventor previously demonstrated that the pharmacological inhibition of GSK-3β by SB216763 limits significantly the infection by HSV-1 (Li Puma et al., Glia, 69: 201-215, 202; doi: 10.1002/glia.23895). Several studies, moreover, demonstrated the antiviral effectiveness of lithium, although at concentrations well above the toxicity limit of such cation (>10 mM) at systemic level. Within the antiviral action, it was demonstrated that glutathione, in turn, exerts an important antiviral action by contrasting the oxidative stress produced after the viral invasion.

Based upon these studies the effectiveness of LiG-AuNPs in exerting an antiviral action in experimental models of infection by in vitro HSV-1 was then evaluated.

The infection by in vitro HSV-1 provides a “contact” time between the cells and the virus, called “adsorption period”, usually lasting 1 hour, performed at 37° C. in culture means without serum (for example, foetal bovine serum—FBS). During this time range the virus can bind to the cellular membrane and enter the host cell. At the end of the adsorption time, the not adsorbed virus is removed by washing in phosphate buffer (PBS) and the cells are incubated until 24 hours [post infection period (p.i.)] in a culture medium added with 2% FBS to allow the viral replication. At the end of 24 hours the infection effectiveness is determined both by quantification of the viral titre in the extracellular medium through standard assay of the plaques, and by dosing the expression of the viral proteins (through WB and immunofluorescence).

The effectiveness of LiG-AuNPs in inhibiting the infection by HSV-1 was then studied by modulating the experimental conditions to evaluate both the concentration and the effecting administration time of use.

Confluent single-layers of human neuroblastoma cells (SH-SY5Y), epithelial cells of monkey kidney (VERO), and murine cortical astrocytic primary cells, were infected by HSV-1 (with infection multiplicity [MOI] equal to 1) and subsequently analysed 24 hours p.i. under experimental conditions described in details in FIGS. 5 and 6, and specifically: treatment with vehicle or LiG-AuNPs at various concentrations: 0.05 mg/mL, 1 mg/mL and 2 mg/mL; during the virus adsorption phase only, the post-infection phase only or all the infection phases.

Although depending upon the cellular type, the results of these experiments show that the LiG-AuNPs are effective in inhibiting/reducing the infection by in vitro HSV-1, by exerting a greater action when applied during the whole infection phase, even at the lower concentration than 0.05 mg/mL (0.15 mEq extracellular Li⁺; FIG. 6E).

For some experiments (i.e., immunofluorescence) the NaG-AuNPs were used as control of LiG-AuNPs (FIG. 5). It is important to remind that although they do not contain lithium, the NaG-AuNPs are however protected by glutathione which could exert a weak antiviral action, by contrasting the oxidative stress produced by the viral invasion.

### Example 1. Method for Preparation of Gold Nanoparticles Coated with Lithiated Reduced Glutathione (LiG-AuNPs)

By way of example a procedure is described, generally valid, scalable and modulable at will, for the synthesis of gold nanoparticles coated with lithiated reduced glutathione (LiG-AuNPs). The type and the amounts of reagents and solvents, the instruments and experimental conditions described hereinafter are not to be meant as limiting the current patent proposal.

The method for the preparation of LiG-AuNPs comprises the following detailed steps:
1. A glass reaction flask with round bottom having volume of 100 mL, provided with a magnetic anchor for stirring, is loaded at the room temperature and atmospheric pressure with the following chemical compounds: tetrachloroauric acid trihydrate (HAuCl₄·3H₂O, number CAS: 16961-25-4, in the amount of 0.333 g corresponding to 0.846 mmol), methanol (number CAS: 67-56-1, in the amount of 27.8 mL), water (in the amount of 22.2 mL), reduced glutathione (GSH, number CAS: 70-18-8, in the amount of 0.581 g corresponding to 1.89 mmol) and lithium hydroxide monohydrate (LiOH·H₂O, number CAS 1310-66-3, in the amount of 0.388 g corresponding to 9.25 mmol). The addition of reduced glutathione caused the turbidity of the reaction means; turbidity which disappears quickly, in few seconds, after adding lithium hydroxide, by allowing to obtain a colourless limpid solution.
II. The solution obtained in step (1) is transferred, at room temperature and atmospheric pressure, in a glass reaction flask with round bottom having a volume of 3 L provided with a magnetic anchor for stirring.
III. The solution obtained in step (II), at room temperature and atmospheric pressure, is diluted with 260 mL of methanol and 760 mL of water.
IV. The solution obtained in step (Ill), at room temperature and atmospheric pressure, is treated quickly with an aqueous solution of just prepared sodium borohydride (NaBH₄, number CAS: 16940-66-2; in the amount of 0.145 g corresponding to 3.83 mmol dissolved in 15 mL of deionized water) under vigorous stirring at room temperature. The addition of the reducing agent causes the formation of a dark brown colloidal suspension (see FIG. 1).
V. The intermediate obtained in step (IV) is kept under magnetic stirring for 48 hours at room temperature and atmospheric pressure, after that lithium chloride (LiCl, number CAS: 7447-41-8, in the amount of 15.38 g corresponding to 0.363 mol) and methanol (700 mL) are added.
VI. The colloidal suspension obtain in step (V) is transferred in a glass cone of Imhoff type, where the particles LiG-AuNPs are subjected to precipitation in about 48-72 hours. The supernatant is then moved away, the particles are transferred in 50 mL centrifuge tubes and subjected to centrifugation (6500 rpm for 10 min). The supernatant is delicately removed from the centrifuge tube and the particles vacuum dried. The procedure allows to obtain about 0.25 g of LiG-AuNPs.

1-28. (canceled)

29. A composition comprising one or more glutathione and Li⁺ coated gold nanoparticles having a diameter from 0.1 to 100 nm.

30. The composition of claim 29, wherein the gold on each nanoparticle is present in an amount from 40 to 60% w/w, the glutathione on each nanoparticle is present in an amount from 20 to 30% w/w, and the lithium on each nanoparticle is present in an amount from 0.1 to 10% w/w.

31. The composition of claim 29, wherein the one or more nanoparticles are prepared with a method comprising:
i) preparing a mixture of
a gold precursor in a concentration from 0.0001M to 10M;
at least a polar solvent;
glutathione in a molar ratio with respect to the gold precursor from 0.1:1 to 100:1; and
a basic lithium compound in a molar ratio with respect to the gold precursor from 10:0.01 to 0.01:10,
at a temperature ranging from −20° to 120° C. in an environment equipped with a stirring and shaking system to obtain a clear colourless solution;
ii) diluting in a range from 1:0 to 1:1000 the solution obtained in step i) with at least a polar solvent;
iii) adding to the diluted solution of step ii) a reducing agent of the gold precursor in a molar ratio with respect to the gold precursor from 1:1 to 100:0.01 to obtain a colloidal suspension of gold nanoparticles at a temperature ranging from −20° to 120° C. in an environment equipped with a stirring and shaking system;
iv) adding a lithium salt in a molar ratio with respect to the gold precursor from 0.1:1 to 100:0.001;
v) diluting in a range from 1:0 to 1:1000 the solution obtained in step iv) with at least a polar solvent; and
vi) purifying the nanoparticles obtained in steps iv)-v).

32. The composition of claim 31, wherein:
step iii) is carried out with constant stirring for a time ranging from 1 to 120 hours; and/or
step vi) is carried out by removal of the supernatant liquid by sedimentation, centrifugation, dialysis, filtration, centrifugal ultrafiltration, or any combination thereof, and subsequently drying in vacuum, under air, by heating, by gas flow, or any combination thereof.

33. The composition of claim 31, wherein:
the gold precursor is selected from gold halides, gold chalcogens, gold pycnogens, gold crystallogens, gold complexes, gold clusters (I or III), organoauric compounds, or any mixture thereof,
the polar solvent is at least one of:
an aprotic polar solvent selected from acetone, acetonitrile, tetrahydrofuran, dioxane, dimethyl sulfoxide, dimethylformamide, a peralkylated urea, and hexamethylphosphoramide;
a polar protic solvent selected from water, alcohol, carboxylic acid, amine, hydroalcoholic solution, a sulfonated compound, and a nitrated compound; and
a mixture thereof;
the basic lithium compound is selected from anhydrous or monohydrate lithium hydroxide, lithium oxide, lithium hydride, lithium alkoxides, lithium amides, lithium carbonate, lithium bicarbonate, lithiated Zintl compounds, metallic lithium, lithium/ammonia solutions, lithium amalgam, lithiated anion resins, lithium phosphates, lithium sulfates, lithium carboxylic compounds, lithium tetraborate, lithiated borates, lithium fluoride, lithium hypochlorite, lithium chlorite, lithium oxyanions, organolytic compounds, and mixtures thereof; and
the lithium salt is an inorganic or organic lithium compound.

34. The composition of claim 31, wherein the gold precursor is tetrachloroauric acid in trihydrate form.

35. The composition of claim 31, wherein the polar solvent is methanol, a hydroalcoholic solution of methanol, or a mixture comprising water and methanol.

36. The composition of claim 31, wherein the glutathione is glutathione in reduced form (CAS: 70-18-8).

37. The composition of claim 31, wherein the reducing agent of the gold precursor is selected from sodium borohydride, lithium borohydride, and lithium aluminium hydride.

38. The composition of claim 31, wherein the lithium salt is an inorganic or organic lithium compound.

39. The composition of claim 29, comprising a plurality of the nanoparticles in the form of an aggregate, wherein the aggregate has a diameter from 0.1 to 5000 nm.

40. The composition of claim 39, wherein the aggregate is prepared with a method comprising a step of dispersing the particles in a solvent selected from deionized water, an alcohol, a hydroalcoholic solution, a pharmaceutically acceptable solution, and a pharmaceutically acceptable suspension for a time of at least one minute at a temperature ranging from +4 to 56° C. in such an amount as to obtain a concentration of the nanoparticles from 0.00001 to 1000 mg/mL.

41. The composition of claim 31, comprising a plurality of the nanoparticles in the form of an aggregate, wherein the aggregate has a diameter from 0.1 to 5000 nm.

42. The composition of claim 41, wherein the aggregate is prepared with a method comprising a step after step vi) of dispersing the particles in a solvent selected from deionized water, an alcohol, a hydroalcoholic solution, a pharmaceutically acceptable solution, and a pharmaceutically acceptable suspension for a time of at least one minute at a temperature ranging from +4 to 56° C. in such an amount as to obtain a concentration of the nanoparticles from 0.00001 to 1000 mg/mL.

43. The composition of claim 42, wherein the solvent is deionized water at a pH of 5.5 to 6.5.

44. The composition of claim 29, comprising:
aggregates of the glutathione and Li⁺ coated nanoparticles;
a solvent selected from deionized water, an alcohol, a hydroalcoholic solution, a pharmaceutically acceptable solvent, and a pharmaceutically acceptable suspension; and
at least a pharmaceutically acceptable excipient and/or carrier.

45. A method of treating a disease, comprising administering the glutathione and Li⁺ coated gold nanoparticles of claim 29 or an aggregate thereof in a therapeutically effective dose to a patient in need thereof.

46. The method of treatment of claim 45, wherein the disease is related to the activation or positive modulation of Glycogen Synthase Kinase-3 (GSK-3) and is an infectious disease, an infection caused by DNA or RNA viruses, a neurodegenerative disease, or a mood disorder.

47. The method of claim 45, wherein the disease is selected from Alzheimer's disease, Parkinson's disease, Huntington's disease, a tauopathy, Coronavirus infection, Orthomyxovirus infection, Filovirus infection, Flavivirus infection, Hepadnavirus infection, Hepevirus infection, Herpesvirus infection, Papillomavirus infection, Pneumovirus infection, Poxivirus infection, Rhinovirus infection, Reovirus infection, Togavirus infection, and a flu virus infection.

48. The method of claim 45, wherein the infection is a Herpes Simplex type 1 (HSV-1) invection, a SARS-CoV-1 infection, or a SARS-CoV-2 infection.
